(19) United States
(12) Patent Application Publication
(10) Pub. No.: US 2013/0070894 A1
(43) Pub. Date: Mar. 21, 2013

(54) SECTIONAL RADIOGRAPHIC APPARATUS FOR BREAST EXAMINATION

(71) Applicant: Hiromichi Tonami, Otokuni-gun (JP)

(72) Inventor: Hiromichi Tonami, Otokuni-gun (JP)

(21) Appl. No.: 13/235,379

(22) Filed: Sep. 17, 2011

(51) Int. Cl. A61B 6/04

(52) U.S. Cl. 378/37

(57) ABSTRACT

A sectional radiographic apparatus for breast examination includes a detector ring with radiation detectors arranged arcuately for receiving a breast of a patient, each of the radiation detectors including a scintillator for converting radiation into light, and a photodetector for detecting the light, a moving device for moving the detector ring along a direction of a central axis of the detector ring relative to the breast of the patient, and a movement control device for controlling the moving device.

### BACKGROUND OF THE INVENTION

(1) Field of the Invention

This invention relates to a sectional radiographic apparatus for breast examination, which detects annihilation radiation pairs emitted from a patient to image a distribution of a radioactive drug in the patient. More particularly, the invention relates to a sectional radiographic apparatus for breast examination, having a detector ring with radiation detectors arranged in a C-shape or in an annular shape (O-shape).

(2) Description of the Related Art

Medical equipment includes a sectional radiographic apparatus for imaging a distribution of a radioactive drug. A specific construction of such a sectional radiographic apparatus will be described. As shown in FIG. 9A, a conventional sectional radiographic apparatus has a detector ring 62 with radiation detectors 51 arranged in an annular shape for detecting radiation (see Japanese Unexamined Patent Publication No. 2004-279057). This detector ring 62 detects a radiation pair (annihilation radiation pair) emitted in opposite directions from a radioactive drug in a patient.

A construction of the detector ring 62 will be described. The detector ring 62 has a thickness corresponding to three radiation detectors 51 in z-direction as shown in FIG. 9A. The detector ring 62 is formed of the radiation detectors 51 all of which are the same type.

A method of examination using the sectional radiographic apparatus will be described. A patient injected with a radioactive drug is introduced into the detector ring 62. The sectional radiographic apparatus images a distribution of the radioactive drug in the part of the patient introduced into the detector ring 62. Thus, the space inside the detector ring 62 serves as the radiographic field of view of the sectional radiographic apparatus.

The sectional radiographic apparatus includes a mammographic apparatus for examining the breasts of a patient. Such a mammographic apparatus images a distribution of a radioactive drug in a breast of the patient introduced into the detector ring 62, the breast of the patient being fitted within the limits of the radiographic field of view of the sectional radiographic apparatus.

However, the conventional sectional radiographic apparatus has the following problem.

In the conventional sectional radiographic apparatus, the detector ring 62 has different portions thereof varied in the sensitivity for detection of annihilation radiation pairs. The conventional sectional radiographic apparatus is constructed without regard to such variations, which lowers the detection sensitivity of the sectional radiographic apparatus.

Such a problem will particularly be described. FIG. 9 shows a state where a patient M is actually introduced into the detector ring 62. The detector ring 62 can easily detect an annihilation radiation pair generated at a point P1 adjacent the middle in the z-direction of the detector ring 62 as in FIG. 9A. In order to detect the annihilation radiation pair, the detector ring 62 needs to detect both parts of the radiation forming the annihilation radiation pair. The annihilation radiation pair generated at the point P1 adjacent the middle has a high possibility of falling on the detector ring 62, even if their directions of movement are inclined in the z-direction relative to the detector ring 62. Thus, an annihilation radiation pair as indicated by the arrows in FIG. 9A is also detectable.

However, it is difficult for the detector ring 62 to detect an annihilation radiation pair generated at a point P2 adjacent a papillary area of the patient M as in FIG. 9B. Although one of the annihilation radiation pair generated at the point P2 adjacent the papillary area falls on the detector ring 62 as in FIG. 9B, the other flies away to the exterior of the detector ring 62, without falling on the detector ring 62. Such an annihilation radiation pair is undetectable with the detector ring 62. Actually, the annihilation radiation pairs shown in FIGS. 9A and 9B tend to fall on the detector ring 62 at the same angle. Nevertheless, the pair in FIG. 9A is detectable while the pair in FIG. 9B is undetectable.

That is, the detector ring 62 has different levels of sensitivity for detection of annihilation radiation pairs according to different sites. FIG. 10A illustrates detection of annihilation radiation pairs from site Pa with the highest level of detection sensitivity of the detector ring 62. Widths W1 and W2 in the z-direction from ends of the detector ring 62 to the site Pa are sufficiently large. Therefore, the annihilation radiation pairs detectable with the detector ring 62 can have various directions of movement as indicated by arrows in FIG. 10A.

FIG. 10B illustrates detection of annihilation radiation pairs from site Pb with nearly the lowest level of detection sensitivity of the detector ring 62. Width W3 in the z-direction from one end of the detector ring 62 to the site Pb is sufficiently large. However, width W4 in the z-direction from the other end of the detector ring 62 to the site Pb is small. Since the directions of movement of the annihilation radiation pairs detectable with the detector ring 62 are dependent on the smaller width W4, the directions of movement of the annihilation radiation pairs detectable with the detector ring 62 are limited as in FIG. 10B. To summarize the characteristics of the detection sensitivity of such detector ring 62, the detection sensitivity is at the highest level in the middle part in the z-direction of the detector ring 62, and the detection sensitivity becomes the lower the farther away therefrom along the z-direction.

In an actual examination, there is no particular need for the detection sensitivity to be at the highest level in the middle part of the detector ring 62. Nevertheless, according to the conventional construction, the sensitivity for detection of radiation is especially high in the middle part of the detector ring 62. It is convenient if a portion for which tomography is carried out (site of interest) is located in the middle part of the detector ring 62. However, when the site of interest deviates from the middle part of the detector ring 62, sectional images must be generated by a portion of the detector ring 62 low in the sensitivity of radiation detection.

### SUMMARY OF THE INVENTION

This disclosure has been made having regard to the state of the art noted above, and its object is to provide a sectional radiographic apparatus for breast examination which can carry out radiation detection with high sensitivity according to purposes of examination.

The above object is fulfilled, according to this invention, by a sectional radiographic apparatus for breast examination comprising a detector ring, with radiation detectors arranged arcuately, for receiving a breast of a patient, each of the radiation detectors including a scintillator for converting radiation into light, and a photodetector for detecting the light; a moving device for moving the detector ring along a direction of a central axis of the detector ring relative to the breast of the patient; and a movement control device for controlling the moving device.

According to this invention, the detector ring can be moved along the direction of the central axis relative to the breast of the patient. A middle part of the detector ring in the direction of the central axis is a portion which can detect annihilation radiation ray pairs with the highest sensitivity. According to this invention, this high sensitivity portion of the detector ring can be moved relative to the breast of the patient. A sectional position from which sectional images are to be acquired varies with a purpose of examination. Since a site of interest of the patient can be located in the high sensitivity portion of the detector ring according to this invention, the sectional radiographic apparatus for breast examination can acquire sectional images well suited for diagnosis.

It is preferred that the above sectional radiographic apparatus for breast examination further comprises an input device for inputting instructions of an operator, wherein the movement control device, moves a middle part of the detector ring in the direction of the central axis to a site of interest to be radiographed of the patient in response to the instructions of the operator.

The above construction includes the input device for the operator to input instructions. This enables the operator to locate a site of interest of the patient in the middle part (high sensitivity portion) on the central axis of the detector ring reliably.

It is also preferred that the above sectional radiographic apparatus for breast examination further comprises a radiation-blocking shield disposed to cover one end of the detector ring through which the breast of the patient is introduced into the detector ring, wherein the moving device is arranged to move the detector ring and the shield together.

The above construction includes the shield for blocking radiation generating from regions other than the breast of the patient and traveling toward the detector ring when generating sectional images. This shield is constructed movable in the direction of the central axis following movement of the detector ring. Since the shield constantly covers one end of the detector ring regardless of the position in the direction of the central axis of the detector ring, unwanted radiation can be blocked reliably.

In the above sectional radiographic apparatus for breast examination, the detector ring, preferably, is C-shaped.

The above construction can provide a sectional radiographic apparatus for breast examination which can introduce the breast of the patient deep into the detector ring. This is because, with the C-shaped array, a cutout of the detector ring can receive an arm of the patient.

In the above sectional radiographic apparatus for breast examination, the detector ring, preferably, is O-shaped.

The above construction can provide the sectional radiographic apparatus for breast examination having high sensitivity for detection. If a site of interest of the patient is surrounded with no blind sport by the detector ring, detectable annihilation radiation will increase by a corresponding amount. Therefore, the sectional radiographic apparatus for breast examination provided can acquire sectional images with increased sharpness.

### BRIEF DESCRIPTION OF THE DRAWINGS

For the purpose of illustrating the disclosure, there are shown in the drawings several forms which are presently preferred, it being understood, however, that the disclosure is not limited to the precise arrangement and instrumentalities shown.

FIG. 1 is a functional block diagram illustrating a construction of a sectional radiographic apparatus for breast examination according to Embodiment 1;

FIG. 2 is a perspective view illustrating a construction of a radiation detector according to Embodiment 1;

FIG. 3 is a plan view illustrating a construction of a detector ring according to Embodiment 1;

FIG. 4 is a sectional view illustrating a construction of a moving mechanism according to Embodiment 1;

FIG. 5 is a sectional view illustrating the construction of the moving mechanism according to Embodiment 1;

FIG. 6 is a sectional view illustrating an effect of movement of the detector ring according to Embodiment 1;

FIG. 7 is a sectional view illustrating the effect of movement of the detector ring according to Embodiment 1;

FIG. 8 is a plan view illustrating a construction according to one modification of this invention;

FIG. 9A and FIG. 9B are sectional views illustrating a construction of a conventional sectional radiographic apparatus for breast examination; and

FIG. 10A and FIG. 10B are sectional views illustrating the construction of the conventional sectional radiographic apparatus for breast examination.

### DESCRIPTION OF THE PREFERRED EMBODIMENTS

### Construction of Sectional Radiographic Apparatus

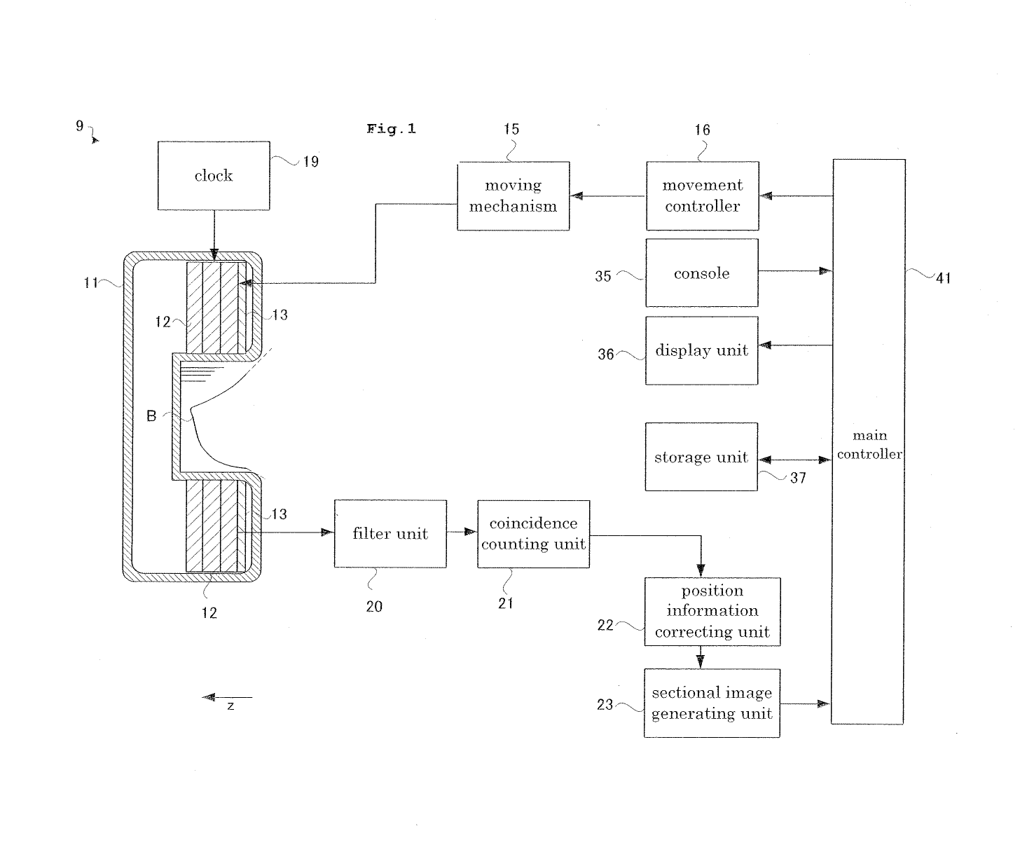

An embodiment of a sectional radiographic apparatus for breast examination according to this disclosure will be described hereinafter with reference to the drawings. Gamma rays in Embodiment 1 are an example of the radiation in this disclosure. The construction in Embodiment 1 is a mammographic apparatus for breast examination, and description will be made by expressing this as the sectional radiographic apparatus as appropriate. FIG. 1 is a functional block diagram illustrating a specific construction of the sectional radiographic apparatus according to Embodiment 1. The sectional radiographic apparatus 9 according to Embodiment 1 includes a gantry 11 into which a breast of a patient is introduced in z-direction, and a detector ring 12 in an annular shape mounted in the gantry 11 into which the breast of the patient is introduced in the z-direction. The detector ring 12 has an inner hole in a cylindrical shape (shape of an octagonal prism to be precise) extending in the z-direction. Therefore, the detector ring 12 itself also extends in the z-direction. The area in the inner hole of the detector ring 12 serves as the radiographic field of view for generating sectional images with the sectional radiographic apparatus 9. The gantry 11 in Embodiment 1 has the characteristic shape, which will be described hereinafter.

A shielding plate 13 is formed of tungsten, for example. Since a radioactive drug is present also in parts other than a breast B of the patient, annihilation gamma ray pairs generate also from such parts. However, the annihilation gamma ray pairs generating from such parts other than the site of interest and incident on the detector ring 12 will become obstructive to sectional image radiography. Therefore, the ring-shaped shielding plate 13 is provided to cover one end of the detector ring 12 close to the patient M in the z-direction. The shielding plate 13 is attached to the detector ring 12.

A moving mechanism 15 is provided to move the detector ring 12 in the z-direction relative to the gantry 11. The detector ring 12 is thereby movable in the z-direction relative to the breast B of the patient. The detector ring 12 and shielding plate 13 are movable together in the z-direction by the moving mechanism 15. A movement controller 16 controls the moving mechanism 15.

A clock 19 outputs time information in serial numbers to the detector ring 12. Detection data outputted from the detector ring 12 includes time information indicating points of time at which gamma rays are detected. The data is inputted to a filter unit 20 described hereinafter.

The construction of the detector ring 12 will be described. The detector ring 12 has three radiation detectors 1 arranged in the z-direction (see FIG. 1).

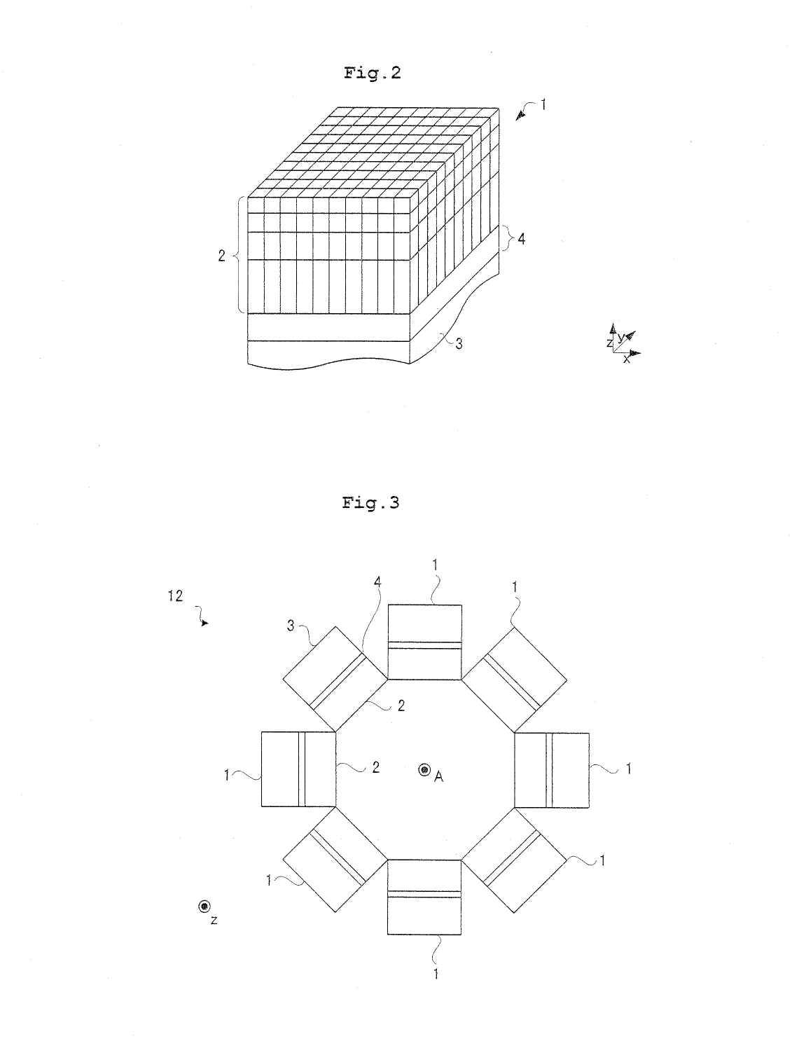

The construction of radiation detectors 1 will be described briefly. FIG. 2 is a perspective view illustrating the construction of the radiation detectors according to Embodiment 1. As shown in FIG. 2, each radiation detector 1 includes a scintillator 2 for converting radiation into light, and a photodetector 3 for detecting the light. A light guide 4 is interposed between the scintillator 2 and photodetector 3 for receiving and delivering the light.

The scintillator 2 is constructed of scintillator crystals arranged in three dimensions. The scintillator crystals are formed of Lu_{2(1-X)}Y_{2X}SiO₅ (hereinafter referred to as LYSO) with Ce diffused. The photodetector 3 can determine positions of occurrence of light, i.e. which scintillator crystals emit light, and can determine also intensity of the light and time at which the light occurs. The construction of the scintillator 2 in Embodiment 1 is only an example that can be employed. Therefore, the construction of this disclosure is not limited to this.

The construction of the detector ring 12 will be described in greater detail. FIG. 3 is a plan view of the detector ring 12 seen from the z-direction. The detector ring 12 is constructed of eight radiation detectors 1 arranged in a circle. The inner hole of the detector ring 12 is in the shape of an octagonal prism. Moreover, all inner walls of the detector ring 12 are covered by the scintillators 2. When the detector ring 12 has a central axis A (axis of rotational symmetry of the inner walls of the detector ring 12) extending along the z-direction, the central axis A is located at the central point of the inner hole of the detector ring 12. When the respective radiation detectors 1 are seen from this central axis A, all the scintillators 2 of the radiation detectors 1 face the central axis A. Each radiation detector 1, as seen from the central axis A, has the light guide 4 and photodetector 3 on the back side of the scintillator 2.

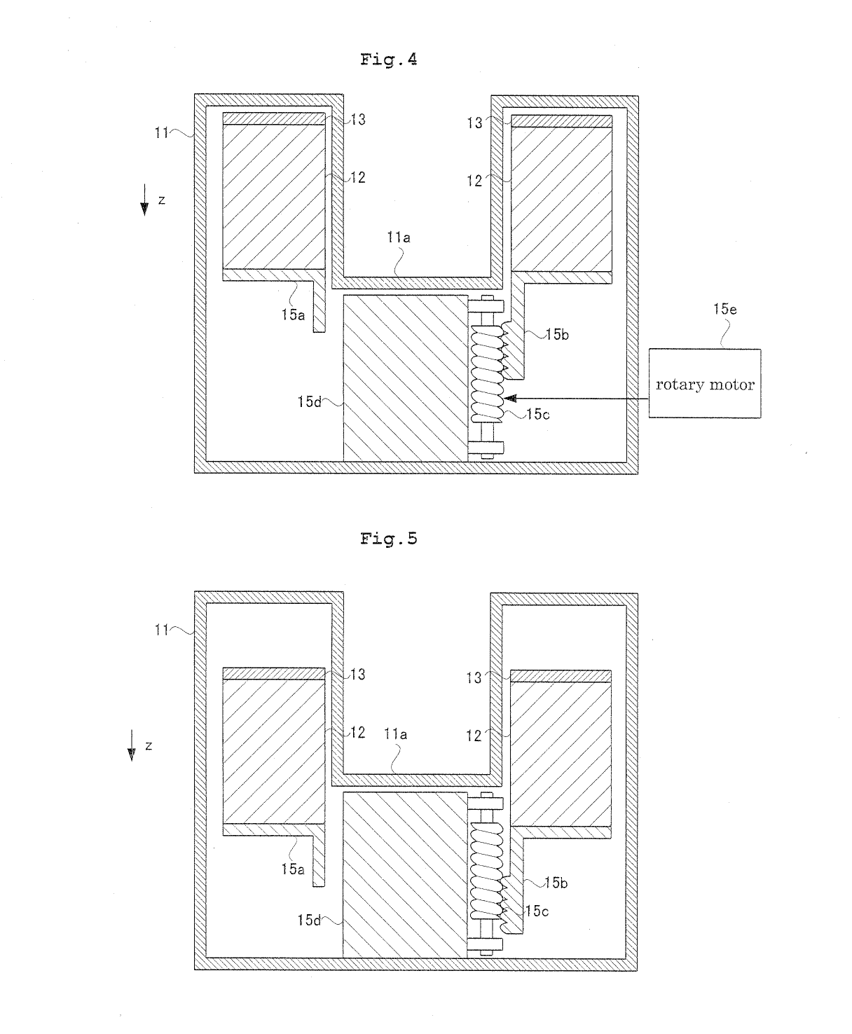

A specific construction of the moving mechanism 15 will be described. As shown in FIG. 4, the moving mechanism 15 is mounted inside the gantry 11. Although the gantry 11 in Embodiment 1 is shaped to form an opening for accommodating the detector ring 12, the inner space of the gantry 11 for receiving the patient M extends in the z-direction is closed halfway instead of penetrating the gantry 11. Therefore, the gantry 11 is constructed to have a recess for receiving the patient M. The detector ring 12 is disposed to cover the wall surfaces except a closed end 11a of this recess.

A ring-shaped support plate 15a is disposed on the surface opposite from the surface of the detector ring 12 covered with the shielding plate 13. The support plate 15a is attached to the detector ring 12. The support plate 15a has a rack 15b extending in the z-direction away from the detector ring 12.

A support base 15d is disposed inside the gantry 11. The support base 15d is joined to the gantry 11 to extend in the z-direction from the surface opposite the side of the gantry 11 having the opening for introducing the patient M toward the closed end 11a of the recess of the gantry 11. The support base 15d has a worm gear 15c extending in the z-direction and rotatable about a base axis parallel to the z-direction. To be exact, the worm gear 15c is rotatably supported by two bearing bars extending from the support base 15d.

The worm gear 15c and rack 15b are in mesh with each other. Rotation of the worm gear 15c moves the rack 15b in the z-direction. This moves the support plate 15a, detector ring 12, and shielding plate 13 together in the z-direction. The worm gear 15c is rotated by a rotary motor 15e. This rotary motor 15e is controlled by the movement controller 16.

When the worm gear 15c is rotated from the state in FIG. 4, the position of the detector ring 12 relative to the gantry 11 can be changed in the z-direction as in FIG. 5. In this way, the detector ring 12 is movable relative to the breast of the patient in the gantry 11.

A coincidence counting unit 21 (see FIG. 1) receives detection data outputted from the detector ring 12 via the filter unit 20. Two gamma rays incident on the detector ring 12 at the same time are an annihilation gamma ray pair resulting from the radioactive drug in the patient. The coincidence counting unit 21 counts the number of times annihilation gamma ray pairs are detected by every combination of two of the scintillator crystals forming the detector ring 12, and outputs the results to a sectional image generating unit 23. Positional relationships between the scintillator crystals in coincidence counting indicate the positions and directions in which the annihilation gamma ray pairs fall on the detector ring 12, and are used in mapping of the radioactive drug. The number of times of annihilation gamma ray pair detection and the energy intensity of annihilation radiation which are stored for every combination of the scintillator crystals indicate variations in generation of the annihilation gamma ray pairs in the patient, which are also used in mapping of the radioactive drug. The time information applied to the detection data by the clock 19 is used by the coincidence counting unit 21 in determining coincidences of the detection data.

The filter unit 20 (see FIG. 1) is provided in order not to output unwanted data from the detector ring 12 to the coincidence counting unit 21. The coincidence counting unit 21 must handle a vast amount of data, and therefore is liable to be heavily loaded. The filter unit 20 can thin out the detection data to reduce the load on the coincidence counting unit 21. For example, the filter unit 20 discards all detection data obtained when the patient M is not inserted in the detector ring 12, and such data is not inputted to the coincidence counting unit 21.

The detector ring 12 moves in the z-direction relative to the breast of the patient M, which will result in a shift of the positional relationship between the patient M and detector ring 12 during examination. A position information correcting unit 22 (see FIG. 1) is provided to correct this shifting. The position information correcting unit 22 receives signals from the movement controller 16 indicating states of movement of the detector ring 12. Based on these signals, the position information correcting unit 22 corrects position information components of the coincidence counting data outputted from the coincidence counting unit 21. Specifically, the position information correcting unit 22 shifts the position information components of the coincidence counting data in the z-direction to follow the movement in the z-direction of the detector ring 12.

The sectional image generating unit 23 receives data relating the generating positions and radiation intensity of annihilation gamma ray pairs outputted from the coincidence counting unit 21, and generates sectional images by spatially mapping the generating positions of annihilation radiation. The sectional images at this time are axial images showing the breast B of the patient cut into round slices, for example.

A display unit 36 displays the sectional images generated by the sectional image generating unit 23. A console 35 is provided for the operator to input various instructions and data to the sectional radiographic apparatus 9. A storage unit 37 stores all of the data produced by operation of each component and parameters referred to for operation of each component, such as the detection data outputted from the detector ring 12, coincidence counting data produced by the coincidence counting unit 2, correction data outputted from the position information correcting unit 22, sectional images and so on.

The sectional radiographic apparatus 9 includes a main controller 41 for performing overall control of the various components. This main controller 41 is constructed of a CPU for executing various programs to realize the respective components 16, 19, 20, 21, 22 and 23. Alternatively, the above components may be realized by being divided into control devices which take charge thereof.

### <Operation of Sectional Radiographic Apparatus>

Next, operation of the sectional radiographic apparatus according to Embodiment 1 will be described. First, the radioactive drug is injected into the patient M. Upon lapse of a predetermined time from this point of time a breast B of the patient is inserted in the detector ring 12. When the operator instructs detection of annihilation gamma ray pairs through the console 35, the detector ring 12 will start outputting detection data to the filter unit 20. The detection data outputted at this time is a data set linking incident positions of the radiation on the detector ring 12, its energy and incidence time.

The coincidence counting unit 21 carries out coincidence counting of the detection data, and outputs the results to the sectional image generating unit 23. The sectional image generating unit 23 generates sectional images showing the breast B of the patient cut into round slices, which are displayed on the display unit 36.

Assume that, at this time, the operator attempts to acquire more detailed sectional images of the papillary area of the patient. The operator gives instructions through the console 35 to move the detector ring 12, and movement of the detector ring 12 is started in response thereto. Then, the papillary area of the patient is located in the middle part in the z-direction of the detector ring 12.

The detector ring 12 detects annihilation gamma ray pairs emitted from adjacent the papillary area of the patient with high sensitivity for detection. Sectional images generated based on this express a distribution of the radioactive drug more vividly than the sectional images of the papillary area of the patient acquired previously. This completes the operation of the sectional radiographic apparatus 9 according to Embodiment 1.

Finally, the effect of moving the detector ring 12 relative to the patient will be described. FIG. 6 is a schematic view of the detector ring 12. An annihilation gamma ray pair generated from an annihilation point P falls on two different spots on the detector ring 12. The longer the detector ring 12 is, the more easily the annihilation gamma ray pair falls on the detector ring 12, and therefore the higher sensitivity the detector ring 12 has for detection of the annihilation gamma ray pair. It is assumed that, of the opposite ends in the z-direction of the detector ring 12, the first end 12p is the closer to the annihilation point P and the second end 12q the farther. The distance from the annihilation point P to the first end 12p is assumed to be the first distance R1, and the distance from the annihilation point P to the second end 12q the second distance R2.

One of the annihilation gamma ray pair generated from the annihilation point P travels toward the first end 12p, and the other toward the second end 12q. When any one of the annihilation gamma ray pair is not detected by the detector ring 12, a coincidence cannot be counted. Therefore, the detector ring 12 needs to detect both the gamma ray having traveled to the first end 12p and the gamma ray having traveled to the second end 12q. It is easy to detect the gamma ray having traveled to the second end 12q because of the long distance R2. However, it is difficult to detect the gamma ray having traveled to the first end 12p since this gamma ray needs to be detected by one portion of the detector ring 12 at the short distance R1. Therefore, the detector ring 12 has a characteristic that it is the more difficult to detect an annihilation gamma ray pair whose generating position is the closer to an end in the z-direction.

The range of the detector ring 12 which can detect the annihilation gamma ray pair generated from a certain annihilation point P is determined by the first distance R1. Specifically, this range is a range R3 which is twice as long as the first distance R1 of the detector ring 12. The middle in the z-direction of the range R3 is in agreement with the position in the z-direction of the annihilation point P. The smaller the range R3 is, the more difficult it is to detect the annihilation gamma ray pair.

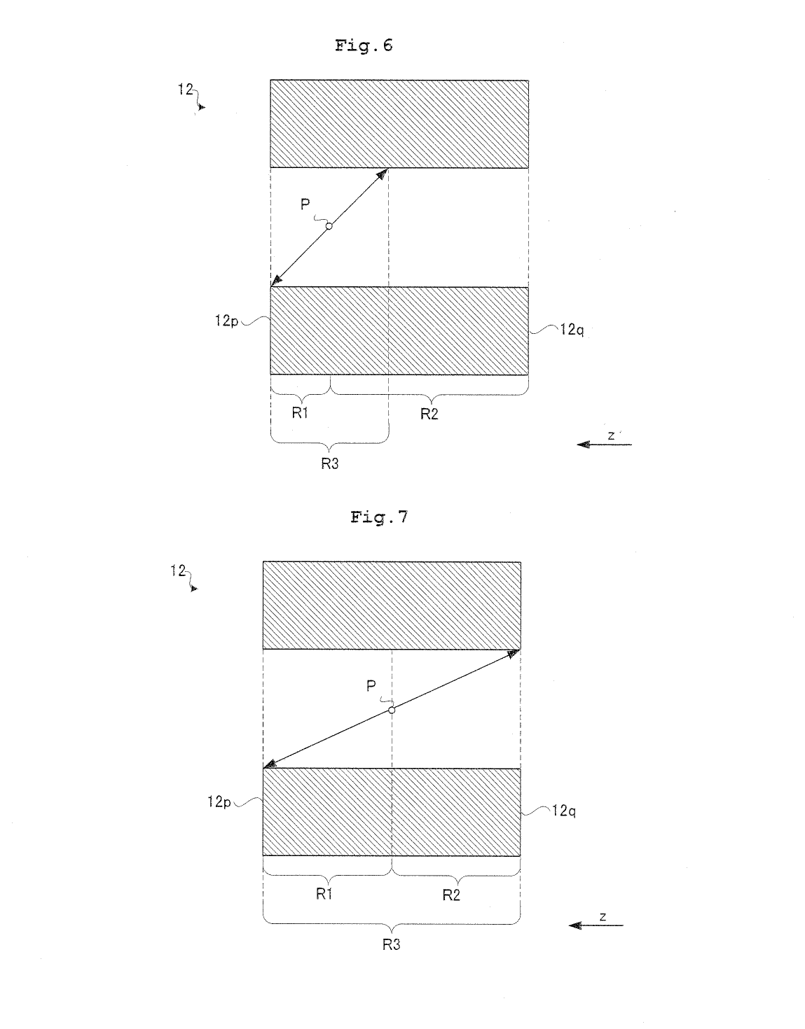

FIG. 6 is a schematic view of a state where the annihilation point P is located adjacent the papillary area of the breast B of the patient in the z-direction of the detector ring 12. In the detector ring 12, the range R3 which can detect the annihilation gamma ray pair emitted from the annihilation point P is narrow. Therefore, under ordinary circumstances, it is difficult to detect such annihilation gamma ray pair. However, according to the construction of Embodiment 1, the detector ring 12 is moved relative to the breast B of the patient. As shown in FIG. 7, the annihilation point P can then be located in a middle part in the z-direction of the detector ring 12. Since the annihilation, point P is located away in the z-direction from both of the opposite ends 12p and 12q, the annihilation gamma ray pair emitted from the annihilation point P are detectable throughout the detector ring 12, resulting in high sensitivity for detection of the annihilation gamma ray pair.

According to the construction of Embodiment 1, as described above, the detector ring 12 can be moved in the z-direction relative to the breast B of the patient. The middle part in the z-direction of the detector ring 12 is a portion which can detect annihilation gamma ray pairs with the highest sensitivity. According to the construction of Embodiment 1, this high sensitivity portion of the detector ring 12 can be moved relative to the breast B of the patient. A sectional position from which sectional images are to be acquired varies with a purpose of examination. Since a site of interest of the patient can be located in the high sensitivity portion of the detector ring 12 according to the construction of Embodiment 1, the sectional radiographic apparatus 9 can acquire sectional images well suited for diagnosis.

The construction of Embodiment 1 includes the console 35 for the operator to input instructions. This enables the operator to locate a site of interest of the patient in the middle part (high sensitivity portion) on the central axis A of the detector ring 12 reliably.

The construction of Embodiment 1 includes the shielding plate 13 for blocking gamma rays generating from regions other than the breast B of the patient and traveling toward the detector ring 12 when generating sectional images. This shielding plate 13 is constructed movable in the z-direction following movement of the detector ring 12. Since the shielding plate 13 constantly covers one end of the detector ring 12 regardless of the position in the z-direction of the detector ring 12, unwanted radiation can be blocked reliably.

The above construction can provide the sectional radiographic apparatus 9 having high sensitivity for detection. That is, the detector ring 12 is shaped cylindrical. If a site of interest of the patient is surrounded with no blind sport by the detector ring 12, detectable annihilation radiation will increase by a corresponding amount. Therefore, the sectional radiographic apparatus 9 provided can acquire sectional images with increased sharpness.

This disclosure is not limited to the foregoing embodiment, but may be modified as follows:

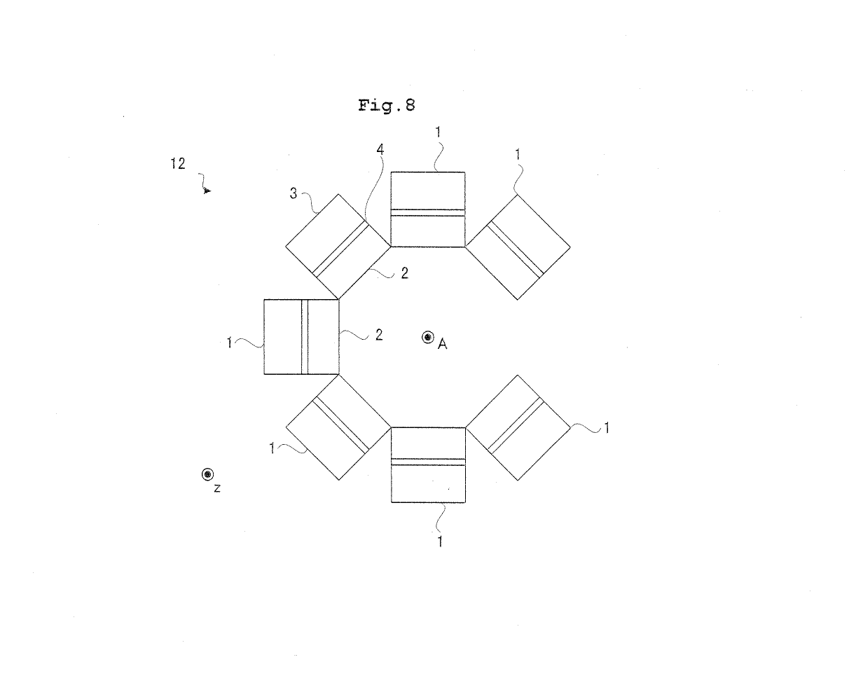

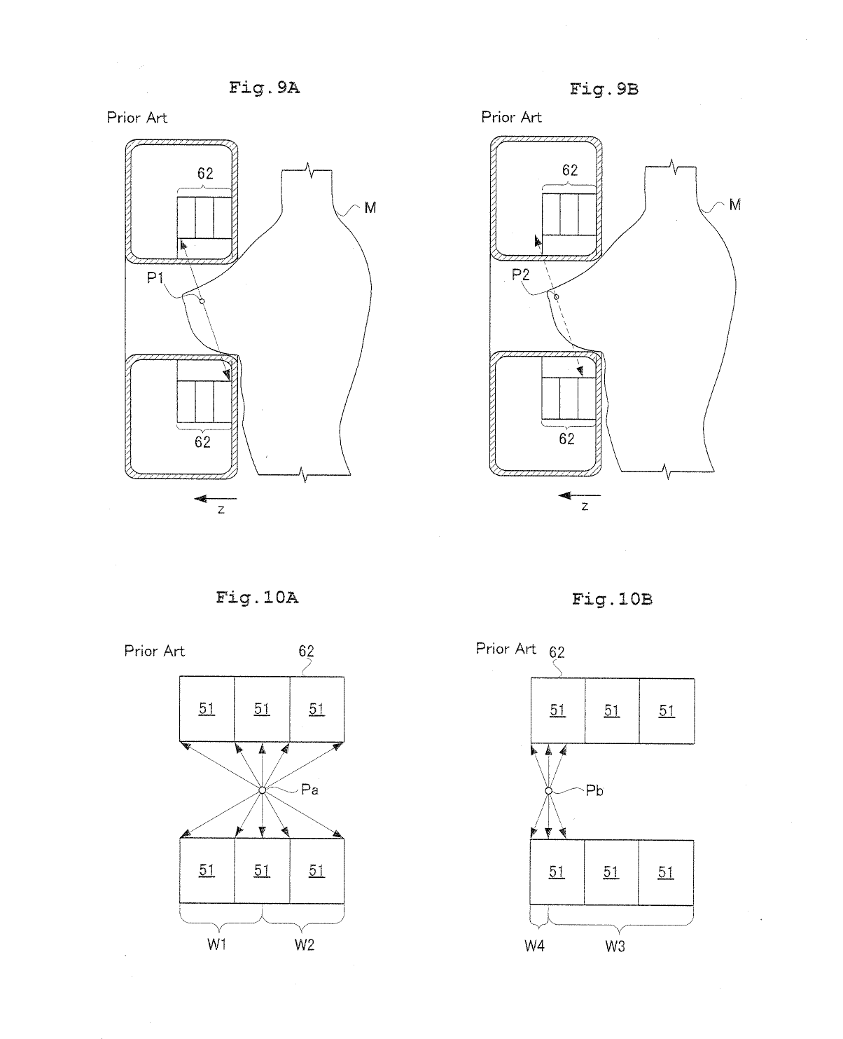

(1) According to the construction of Embodiment 1, the detector ring 12 is ring-shaped. Instead, as shown in FIG. 8, the detector ring 12 may be C-shaped with a cutout. Then the gantry 11 and shielding plate 13 are also C-shaped in accordance with the shape of the detector ring 12. This modification can provide a sectional radiographic apparatus 9 which can introduce the breast B of the patient deep into the detector ring 12. This is because the cutout of the detector ring 12 can receive an arm of the patient.

(2) The scintillator crystals in the foregoing embodiment are formed of LYSO. In this disclosure, the scintillator crystals may be formed of a different material such as GSO (Gd₂SiO₅). This modification can provide a method of manufacturing a less expensive radiation detector.

(3) In the foregoing embodiment, each photodetector includes a photomultiplier tube, but this disclosure is not limited thereto. The photomultiplier tube may be replaced with a photodiode, avalanche photodiode or semiconductor detector.

The present invention may be embodied in other specific forms without departing from the spirit or essential attributes thereof and, accordingly, reference should be made to the appended claims, rather than to the foregoing specification, as indicating the scope of the invention.

1. A sectional radiographic apparatus for breast examination comprising:
a detector ring, with radiation detectors arranged arcuately, for receiving a breast of a patient, each of the radiation detectors including a scintillator for converting radiation into light, and a photodetector for detecting the light;
a moving device for moving the detector ring along a direction of a central axis of the detector ring relative to the breast of the patient;
a movement control device for controlling the moving device; and
a radiation-blocking shield disposed to cover one end of the detector ring through which the breast of the patient is introduced into the detector ring;
wherein the moving device is arranged to move the detector ring and the shield together.

2. The sectional radiographic apparatus for breast examination according to claim 1,
further comprising an input device for inputting instructions of an operator;
wherein the movement control device, moves a middle part of the detector ring in the direction of the central axis to a site of interest to be radiographed of the patient in response to the instructions of the operator.

3. (canceled)

4. The sectional radiographic apparatus for breast examination according to claim 1, wherein the detector ring is C-shaped.

5. The sectional radiographic apparatus for breast examination according to claim 1, wherein the detector ring is O-shaped.

6. (canceled)

7. The sectional radiographic apparatus for breast examination according to claim 2, wherein the detector ring is C-shaped.

8. (canceled)

9. The sectional radiographic apparatus for breast examination according to claim 2, wherein the detector ring is O-shaped.

10. (canceled)

11. A sectional radiographic apparatus for breast examination comprising:
a detector ring, with radiation detectors arranged arcuately, for receiving a breast of a patient, each of the radiation detectors including a scintillator for converting radiation into light, and a photodetector for detecting the light;
a moving device for moving the detector ring within a gantry containing the detector ring in a direction of a central axis of the detector ring, thereby moving the detector ring along the direction of the central axis of the detector ring relative to the breast of the patient; and
a movement control device for controlling the moving device.
